(19) United States
(12) Patent Application Publication
(10) Pub. No.: US 2023/0167173 A1
(43) Pub. Date: Jun. 1, 2023

(54) TREATMENT OF DISEASES WITH CLEVER-1 INHIBITION IN COMBINATION WITH AN INTERLEUKIN INHIBITOR

(71) Applicant: FARON PHARMACEUTICALS OY, Turku (FI)

(72) Inventors: Juho JALKANEN, Mietoinen (FI); Jami MANDELIN, Helsinki (FI); Matti KARVONEN, Turku (FI)

(73) Assignee: FARON PHARMACEUTICALS OY, Turku (FI)

(21) Appl. No.: 17/919,923

(22) PCT Filed: Apr. 19, 2021
(86) PCT No.: PCT/FI2021/050281

§ 371 Date: Oct. 19, 2022

(30) Foreign Application Priority Data
Apr. 20, 2020 (FI) 20205400

(51) Int. Cl. C07K 16/28; C07K 14/555; A61P 35/00

(52) U.S. Cl. CPC C07K 16/28; A61P 35/00; C07K 14/555; C07K 2317/24

(57) ABSTRACT

Use of an agent capable of inhibiting CLEVER-1 expression or binding to CLEVER-1 in combination with an inhibitor of interleukin and/or the respective receptor, and optionally further with an agent capable of binding to interferon-alpha/beta receptor (IFNAR) in a treatment of diseases.

### FIELD OF THE INVENTION

The present invention relates to use of an agent capable of inhibiting CLEVER-1 expression or binding to CLEVER-1 in combination with an inhibitor of interleukin and/or the respective receptor in a treatment of diseases. The present invention also relates to a method for monitoring a patient’s response to anti-CLEVER-1 therapy and evaluating the need for combination therapy comprising an inhibitor of interleukin and/or the respective receptor.

### BACKGROUND OF THE INVENTION

Over the past decade the notion of inflammation has moved beyond heat, swelling, pain and redness, we have gained a detailed understand of the cellular pathways and molecular mediators in inflammation are now being applied to areas of research such as cancer, heart disease, auto-immunity and infectious disease [1].

Inflammation is due to a large variety of stimuli such as damaged and dying cells, chemical irritants and pathogens, these responses are critical for an effective immune response during pathogenic invasions. Two key pillars of the inflammatory response are the innate cytokines; interleukins and type 1 interferons [1].

Diseases such as tuberculosis and hepatitis, viral organisms such as influenza and corona viruses as well as cancer, all cause an inflammation around the site of the disease and go through the pillars of inflammation with pro-inflammatory cytokines; interleukins and type 1 interferon are the initiation of the host response [1].

Recently infective organism like the novel Corona virus (SARS-CoV-2) has caused a huge wave of various developmental strategies to tackle both the virus and the downstream afflictions and complications. Serious complications include septic shock, acute respiratory distress syndrome (ARDS), and multi-organ failure (MOF) which are life threatening conditions. ARDS and MOF are critical conditions and the patients with this complication are treated in the ICU where they have limited and no specific treatment for the condition. Patients receive steroids and mechanical ventilation as treatment for the ailment [2]. Increased levels of pro-inflammatory cytokines are associated with poor prognosis in ARDS [3].

Current treatment options such as steroids show no clinical benefit. Whilst steroids had an accelerated resolution of respiratory failure and circulatory shock, they also increased the risk of secondary infections. A common feature seen in sepsis, severe COVID-19 infection and cancer is the exhaustion of the immune system. It has been observed recently in the pandemic of SARS-CoV-2 that exhaustion markers upon T cells is comparable to that seen in cancer and patients with chronic infections [5]-[8].

Cancer cells have a vast number of genetic and epigenetic alterations that generate plenty of tumour-associated antigens for the host immune system, thereby requiring tumours to develop specific immune resistance to the mechanisms of inflammation discussed above.

An important immune resistance mechanism involved in cancer, ARDS, COVID-19 infection and sepsis are the immune-inhibitory pathways, where single molecules may control the immune system activity (termed immune checkpoints) and normally mediate immune tolerance to mitigate collateral tissue damage. Recently most breakthroughs in controlling the immune system activation are due to the discovery, understanding and modulation of cytotoxic T-lymphocyte associated antigen-4 (CTLA-4), programmed cell death protein-1 (PD-1) and its ligand PD-L1. Previous work has shown antibody blockade of CTLA-4 in mouse models of cancer induced antitumor immunity. Further, immune-checkpoint receptors like PD-1 limit T cell effector functions within tissues. By upregulating ligands for PD-1, the tumour cells block antitumor immune responses in the tumour microenvironment [9], [10]. There are many immune checkpoint modulators approved for use in the clinic, starting from metastatic melanoma with efficacious results in about 10-20% of patients to having been tested in other tumours (such as prostate, breast and colorectal cancer) but these regimes remain refractory to them. Patients responding favourably to checkpoint inhibition usually have a pre-existing antitumor immune response, which is characterized by high density of interferon (IFN)-y-producing CD8⁺ T cells [10], [11].

To increase the response rate of tumours to these available drugs, it is theorized that the tumour must be in the inflamed state, hence development of strategies to achieve inflamed tumour state are rational.

Many approaches to achieve this have been tried in the clinic, however they all are based with a chemotherapeutic regimen to induce apoptotic cell death, such as anthracyclins in order to increase the amount of neoantigens for stimulating long lasting immunity against the tumour [12], [13]. During the apoptotic cell death, interleukin expression is increased as a result of inflammatory signals, majorly interleukin 1 (IL-1), interleukin 6 (IL-6) and interleukin 8 (IL-8) and their receptors are prevalent, which is essential for tumour growth and resistance to cell death during apoptotic signals [1], [10]. IL-1, IL-6 and IL-8 have many downstream pathways and in recent years, both have been interesting treatment targets for clinical development but for different reasons. IL-1 activation leads downstream to tumour-necrosis factor-associated factor (TRAF) 6 activation resulting further in nuclear factor kappa-light-chain-enhancer of activated B cells (NF-_{K}B) activation. IL-6 has been targeted to stop downstream Janus kinase (JAK) and the downstream phosphorylation of signal transducer and activator of transcription 3 (STAT3), whereas for tackling IL-8 have involved targeting the two G-protein coupled receptors (CXCR1 and CXCR2) thus stopping the downstream signalling in this pathway.

There are no approved cancer drugs that inhibit IL-1, IL-6 and IL-8 or their receptors. Anti-IL-1 and anti-IL-1 receptor inhibitors are marketed for genetic disorders as well as musculoskeletal disorders. Anti-IL-6 or anti-IL-6 receptor antibodies are marketed as anti-inflammatory drugs for rheumatic diseases.

IL-1, IL-6 and IL-8 are pro-inflammatory cytokines, alongside Type 1 interferons they are major players in the inflammation process. These cytokines bind to receptors abundantly overexpressed on the tumour surfaces as well as tissues associated with chronic infection such as granulomas in tuberculosis or in acute severe disease states such as sepsis and ARDS.

IL-1, IL-6 and IL-8 receptors are also abundant on other cells related to the tumours and inflammation at the tumour microenvironment (TME) such as tumour infiltrating neutrophils and tumour associated macrophages [12]. The TME can be compared to granulomas in Tuberculosis and Hepatitis.

Innate immune cells such as macrophages found in both cancer and chronic infections such as Tuberculosis and hepatitis [7], however, can dampen T cell activation and contribute to tumour progression despite high mutational load in tumour cells. The macrophages that contribute to tumour-related immunosuppression and provide tumour growth supporting signals may be highly eligible candidates for targeted therapies, since these cells are abundantly present in various tumours, they are very plastic and can be converted into pro-inflammatory macrophages supporting T cell activation and tumour or infection rejection [15, 16]. To date, macrophage targeted strategies under clinical development utilize macrophage colony-stimulating factor receptor inhibition to deplete macrophage populations in tumours [17]. However, resistances to these approaches have already been reported [18]. Thus, there is a need to find novel ways to utilize these cells to fight against cancer.

In recent years, increasing attention has been paid to the contribution of scavenger receptors in regulating macrophage responses to different stimuli. CLEVER-1 (also known as Stabilin-1) is a multifunctional molecule conferring scavenging ability on a subset of anti-inflammatory macrophages [19, 20]. In these cells, CLEVER-1 is involved in receptor-mediated endocytosis and recycling, intracellular sorting, and transcytosis of altered and normal self-components. More recently, it has been found that the progression of cancer growth and metastasis is attenuated in Stab1^{-/-} (CLEVER-1 knock out) mice, and in mice treated with anti-CLEVER-1 therapy [20].

### SUMMARY OF THE INVENTION

Now, it has been surprisingly found out that anti-CLEVER-1 treatment in cancer in deeply immunosuppressed cancer patients leads to the activation of the immune system that enables the host immune system to fight against sepsis and complete immune exhaustion. It has also been surprisingly found that anti-CLEVER-1 treatment leads to an anti-tumour response, except when there is an increase in interleukins from the immune response driven by CLEVER-1 inhibition or driven by disease progression and immune resistance. Hence, anti-CLEVER-1 treatment has been found to be beneficial to use together with interleukin inhibition therapy and/or by further inducing the immune response achieved by anti-CLEVER-1 agent by administering type I interferons with CLEVER-1 inhibition in a patient having an increase in interleukin expression levels, such as IL-6 and/or IL-8 despite of anti-CLEVER-1 treatment. Anti-interleukin therapy is inhibiting the interleukin and their respective receptors such as IL-6 or IL-6 receptor (IL-6R), IL-8 or IL-8 receptor (IL-8R), and/or IL-1 or the IL-1 receptors IL-1 Ra and/or IL-1 Rb. This immune response can also be caused by an agent capable of binding to interferon-alpha/beta receptor (IFNAR), such as an exogenous type I interferon in combination with CLEVER-1 inhibition for more effective disease therapy in otherwise unresponsive conditions such as acute respiratory distress syndrome (ARDS), sepsis or cancer.

Especially, it has been found that the combination of an agent capable of inhibiting CLEVER-1 expression or binding to CLEVER-1 and an inhibitor of interleukins and/or their respective receptors is suitable for the treatment of the tumours, chronic infection and acute inflammatory infections leading to immune exhaustion, which are not responsive to a monotherapy of an agent capable of inhibiting CLEVER-1 expression or binding to CLEVER-1. Further, an agent capable of binding to interferon-alpha/beta receptor (IFNAR) can be used in the treatment for inducing the immune response. Anti-IL-1 and/or anti-IL-6 and/or anti-IL-8 treatments and/or activation of type I interferon receptor (IFNAR) are not effective cancer treatment as a monotherapy but have shown activity in other indications. In combination with anti-CLEVER-1 agent they have been found to possess anti-tumour and anti-infective activity.

Therefore, an object of the present invention is to provide a novel treatment for cancer, especially to provide treatment method against tumour types which are currently untreatable or do not provide desired response to anti-CLEVER-1 treatment.

A further object of the present invention is to provide a novel treatment for infectious diseases and their deadly acute disease states, such as sepsis and ARDS, to support the immune response against the causative organism or a later opportunistic infection taking advantage of the exhausted immune system needed to fight the first severe condition.

Further, an object of the present invention is to provide a method for monitoring patient’s response to anti-CLEVER-1 therapy and evaluating the need for combination therapy comprising an inhibitor of interleukin and/or the respective interleukin receptor, when an agent capable of binding to CLEVER-1 has already been administered in a patient.

In order to achieve among others the objects presented above, the invention is characterized by what is presented in the characterizing parts of the enclosed independent claims. Some preferred embodiments of the invention will be described in the other claims.

The embodiments and advantages mentioned in this text relate, where applicable, both to the combination of the said agents, the method as well as to the uses according to the invention, even though it is not always specifically mentioned.

According to a first aspect of the present invention, the present invention concerns a combination of therapeutically effective amounts of:
(a) an agent capable of inhibiting CLEVER-1 expression or binding to CLEVER-1, and
(b) an inhibitor of interleukin and/or the respective interleukin receptor, for use in a treatment of disease selected from the group consisting of cancer, infectious diseases, chronic infection, severe influenza or coronavirus infection, sepsis and acute respiratory distress syndrome (ARDS), wherein the agent capable of inhibiting CLEVER-1 expression or capable of binding to CLEVER-1 is administrated to an individual prior to the administration of an inhibitor of an interleukin(s) and/or an inhibitor of the respective interleukin receptor(s) and an individual to be treated having diagnosed an elevation in interleukin IL-1, IL-6 and/or IL-8 levels after beginning anti-CLEVER-1 treatment (i.e. after beginning of the administration of said agent capable of inhibiting CLEVER-1 expression or capable of binding to CLEVER-1).

Especially, the present invention concerns a combination of therapeutically effective amounts of:
(a) an agent capable of inhibiting CLEVER-1 expression or binding to CLEVER-1, and
(b) an inhibitor of interleukin(s) such as IL-1, IL-6 and IL-8 and/or their respective receptors IL-1 Ra, IL-1 Rb, IL-6R and IL-8R,
for use in a treatment of disease selected from the group consisting of cancer, infectious diseases, chronic infection, severe influenza or coronavirus infection, sepsis and acute respiratory distress syndrome (ARDS) in an individual having diagnosed with an indication which shows high expression of pro-inflammatory cytokines (IL-1, IL-6, IL-8) and/or which is not responsive to anti-CLEVER-1 treatment alone or shows an increase in the level of circulating interleukins during anti-CLEVER-1 treatment. Further, an agent capable of binding to interferon-alpha/beta receptor (IFNAR), such as an exogenous type 1 Interferon can be used in addition of an agent capable of inhibiting CLEVER-1 expression or binding to CLEVER-1 and an inhibitor of interleukin(s), such as IL-1, IL-6 and IL-8 and/or their respective receptors IL-1Ra, IL-1 Rb, IL-6R and IL-8R for inducing the immune response and affecting IL-6 and/or II-8 expression levels.

According to the present invention, an inhibitor of interleukin and/or the respective interleukin receptor is used in combination of anti-CLEVER-1 treatment, or an inhibitor of interleukin and/or the respective interleukin receptor and an agent capable of binding to interferon-alpha/beta receptor (IFNAR) is used in combination of anti-CLEVER-1 treatment.

Responsiveness to anti-CLEVER-1 treatment is typically associated with a decrease in IL-1, IL-6 and IL-8 levels, while non-responsiveness to anti-CLEVER-1 treatment is associated with increased IL-6 and IL-8 plasma/serum levels. Anti-CLEVER-1 treatment leads to an increased infiltration of T cells into tumours and granulomas and in this way decreases IL-1 R and/or IL-6R and/or IL-8R expression for improved therapeutics targeting with either an anti-IL-1 and/or anti-IL-6 and/or IL-8 inhibitor, and/or an agonist of IFNAR. Therefore, the present invention provides improved efficacy of an anti-IL-1 and/or anti IL-6 and/or IL-8 treatment, and/or a type 1 interferon (IFN) when combined with anti-CLEVER-1 treatment targeting to block the negative regulation of T cells in cancer, chronic infection, infectious diseases or other states of immune exhaustion, e.g. in sepsis and ARDS. The exhaustion markers upon T cells observed also recently in COVID-19 infection is comparable to that seen in cancer and patient with chronic infections [5]-[8]. Hence, the combination treatment according to the present invention is also suitable for treatment of severe influenza and corona infections such as novel coronaviruses (Sars-Cov and Sars-Cov2) leading to immune exhaustion. The present invention provides a combined treatment of interleukin inhibition and/or Type 1 Interferon with anti-CLEVER-1 agents for patients requiring the activation of the immune system.

According to one aspect, the present invention provides a method for treating or delaying progression of cancer in an individual comprising administering to the individual a therapeutically effective amount of an agent capable of inhibiting CLEVER-1 expression or binding CLEVER-1 in combination with an inhibitor of Interleukin(s) and/or their respective receptor(s), and optionally further with an agent capable of binding to interferon-alpha/beta receptor (IFNAR) such as a type 1 Interferon.

According to another aspect, the present invention provides a method for treating or preventing chronic infection, infectious diseases or other states of immune exhaustion, e.g. in sepsis and ARDS in an individual comprising administering to the individual a therapeutically effective amount of an agent capable of inhibiting CLEVER-1 expression or binding CLEVER-1 in combination with an inhibitor of Interleukin(s) and/or their respective receptor(s), and optionally further with an agent capable of binding to interferon-alpha/beta receptor (IFNAR) such as a type 1 Interferon.

Further, according to one aspect of the present invention, the present invention provides a method for monitoring a patient’s response to anti-CLEVER-1 therapy and evaluating the need for combination therapy comprising an inhibitor of interleukin and/or the respective interleukin receptor, when an agent capable of binding to CLEVER-1 has been administered in a patient, the method comprising
obtaining a sample from the patient at a first point in time prior to the administration of an agent capable of binding to CLEVER-1 to a patient,
obtaining a sample from the patient at a later point in time after the administration of an agent capable of binding to CLEVER-1 to a patient,
measuring a level of interleukin IL-1, IL-6 and/or IL-8 from the obtained samples,
comparing the level of IL-1, IL-6 and/or IL-8 measured from the sample obtained at a later point of time to the expression level of IL-1, IL-6 and/or IL-8 measured from the sample obtained at a first point of time, wherein an elevation in interleukin IL-1, IL-6 and/or IL-8 levels is an indication for initiation the concomitant administration of IL-1 inhibitor and/or an inhibitor of the respective receptor, IL-6 inhibitor and/or an inhibitor of the respective receptor, IL-8 inhibitor and/or an inhibitor of the respective receptor, or any combination of thereof.

In addition, it has been found that the preferred dose range is 0.3 - 10 mg/kg, preferably 0.3 mg/kg to 3 mg/kg, according to the patient’s body weight, for using a humanized anti-CLEVER-1 antibody, such as bexmarilimab for providing immune stimulation for the treatment of said diseases according to the present invention. Unlike conventional pharmacological disease treatment, which are used at a maximum tolerated dose, anti-CLEVER-1 antibody treatment creates an immune response. With low doses the immune response does not occur, and with high doses the immune system creates new ways to balance out the achieved immune activation, e.g. through the increase of CLEVER-1 expression or secretion of IL-8.

### BRIEF DESCRIPTION OF THE DRAWINGS

FIG. 1. IFNγ, IL-6 and IL-8 changes in serum of patients during anti-CLEVER-1 treatment and comparison between patients that have an anti-tumour response, i.e. stable disease or partial response (SD/PR), compared to patients with progressive disease (PD). An up-regulation of IFNγ, but down regulation of IL-6 and IL-8 are associated with an anti-tumour response.

FIG. 2. IFNγ, IL-6 and IL-8 changes during anti-CLEVER-1 treatment according to different doses. The most favourable immunological responses are seen at doses 0.3 mg/kg, 1 mg/kg and 3 mg/kg.

FIG. 3. Immune reinvigoration of a deeply immunosuppressed cancer patients with anti-CLEVER-1 antibody (FP-1305). This enabled the patient 1 to survive sepsis. Prior to anti-CLEVER-1 treatment the patients’ peripheral blood cells did not response in any way to an LPS stimulus. LPS consists of bacterial fragments. After being dosed with a humanized anti-CLEVER-1 antibody the patients’ blood cells reacted “normally” to an LPS stimulus and produced cytokines that are needed to counterattack an infection. Immune exhaustion into immune activation was achieved. C = treatment cycle, D = day

### DETAILED DESCRIPTION OF THE INVENTION

CLEVER-1 is a protein disclosed in the Patent Publication WO 03/057130, Common Lymphatic Endothelial and Vascular Endothelial Receptor-1. CLEVER-1 (also known as Stabilin-1) is a multifunctional molecule conferring scavenging ability on a subset of anti-inflammatory macrophages [19, 20].

The terms “an agent capable of inhibiting CLEVER-1 expression or capable of binding to CLEVER-1”, “CLEVER-1 inhibitor” and “anti-CLEVER-1 agent” are interchangeable and refers to agents including antibodies and fragments thereof, peptides or the like, which are capable of inhibiting CLEVER-1 expression or binding to CLEVER-1 for blocking the function of CLEVER-1 or blocking the interaction of CLEVER-1 and cells involved with disease etiology. The agent may also be any other inhibitor, such as RNA therapy, small molecule inhibitor or macromolecule having an adequate affinity to bind to CLEVER-1 receptor or capability to reduce its expression and/or to inhibit the protein activity. The term “an antibody, fragment or molecule thereof” is used in the broadest sense to cover any therapeutic agent whether be antibody, fragment or small molecule thereof which are capable to inhibit CLEVER-1 expression or bind CLEVER-1 molecule in an individual. Especially, it shall be understood to include chimeric, humanized or primatized antibodies, as well as antibody fragments and single chain antibodies (e.g. Fab, Fv), so long they exhibit the desired biological activities. Particular useful agents are anti-CLEVER-1 antibodies and fragments thereof. Therefore, according to an embodiment of the present invention the agent capable of inhibiting CLEVER-1 expression or capable of binding to CLEVER-1, i.e. CLEVER-1 inhibitor or anti-CLEVER-1 agent, is selected from the group consisting of an antibody or a fragment thereof, peptide(s), RNA, small molecule or macromolecule and any combination thereof. Anti-CLEVER-1 treatment or anti-CLEVER-1 therapy refers to a treatment or therapy comprising administration of an agent capable of inhibiting CLEVER-1 expression or capable of binding to CLEVER-1.

In an embodiment according to the present invention, an agent capable of inhibiting CLEVER-1 expression or capable of binding to CLEVER-1 comprises a humanized monoclonal anti-CLEVER-1 antibody. In an embodiment of the present invention, the anti-CLEVER-1 antibody is a humanized monoclonal immunoglobulin G4_{K} antibody bexmarilimab (International Nonproprietary Name (INN) as disclosed in WHO Drug Information, Vol. 33, No.4 (2019) as proposed INN and in WHO Drug Information, Vol. 34, No. 3 (2020), pages 699-700 as recommended INN), or bexmarilimab variant or the antibody in a bexmarilimab biosimilar.

A bexmarilimab biosimilar means a biological product which is approved by a regulatory agency in any country for marketing as a bexmarilimab biosimilar. In an embodiment, a bexmarilimab biosimilar comprises a bexmarilimab variant as the drug substance. In an embodiment, a bexmarilimab biosimilar has substantially the same amino acid sequence of heavy and light chains as bexmarilimab. As used herein, a “bexmarilimab variant” means an antibody which comprises sequences of heavy chain and light chain that are identical to those in bexmarilimab, except for having one or more conservative amino acid substitutions at positions that are located outside of the light chain CDRs and/or one or more conservative amino acid substitutions that are located outside of the heavy chain CDRs, e.g. the variant positions are located in the framework regions or the constant region. In other words, bexmarilimab and a bexmarilimab variant comprise identical CDR sequences, but differ from each other due to having a conservative amino acid substitution at other positions in their full-length light and heavy chain sequences. A bexmarilimab variant is substantially the same as bexmarilimab with respect to binding affinity to CLEVER-1.

According to an embodiment of the present invention, a cell line producing the therapeutic anti-CLEVER-1 antibody bexmarilimab (FP-1305) has been deposited on 27 May 2020 under the terms of the Budapest Treaty on the International Recognition of the Deposit of Micro-organisms for the Purposes of Patent Procedure with the DSMZ-German Collection of Microorganisms and Cell Cultures GmbH, Inhoffenstrasse 7B, D-38124 Braunschweig, Germany, and has the accession number DSM ACC3361. The present invention is not to be limited in scope by the culture deposited, since the deposited embodiment is intended as a single illustration of one aspect of the invention and any culture that is functionally equivalent is within the scope of this invention. The deposit of material herein does not constitute an admission that the written description herein contained is inadequate to enable the practice of any aspect of the invention, including the best mode thereof, nor is it to be construed as limiting the scope of the claims to the specific illustration that it represents.

According to the present invention, an agent capable of inhibiting CLEVER-1 expression or capable of binding to CLEVER-1 is used in combination with an inhibitor of interleukin(s) and/or the respective interleukin receptor(s), in the activation of the immune system. In addition, an agent capable of binding to Interferon-alpha/beta receptor IFNAR can be used together with an agent capable of inhibiting CLEVER-1 expression or capable of binding to CLEVER-1 and an inhibitor of interleukin(s) and/or the respective interleukin receptor(s). Especially, said combination(s) are used in the treatment of the individual having diagnosed with an indication which shows high expression of pro-inflammatory cytokines (IL-1, IL-6, IL-8) and/or which is not responsive to anti-CLEVER-1 treatment alone or shows an increase in the level of circulating interleukins during anti-CLEVER-1 treatment. According to the present invention, said combination treatment(s) are used for treatment or prevention of a disease selected from the group consisting of cancer, infectious diseases, chronic infection, severe influenza or coronavirus infection, sepsis, severe influenza or coronavirus infection, acute respiratory distress syndrome (ARDS) and multiorgan failure (MOF).

The term “treatment” or “treating” shall be understood to include complete curing of a disease or disorder, as well as amelioration or alleviation of said disease or disorder. The term “therapeutically effective amount” is meant to include any amount of an agent according to the present invention that is sufficient to bring about a desired therapeutic result.

In an embodiment of the present invention, an inhibitor of interleukin and/or the respective interleukin receptor is selected from the group consisting of IL-1 inhibitor and/or an inhibitor of the respective receptor, IL-6 inhibitor and/or an inhibitor of the respective receptor, IL-8 inhibitor and/or an inhibitor of the respective receptor, or any combination of thereof. In the present invention, an anti-IL-1 and/or anti-IL-6 and/or anti-IL-8 therapy refers to the inhibitors that are capable of blocking the either IL-1/IL-1R or IL-6/IL-6R or IL-8/IL-8R being CXCR1 or CXCR2 signalling pathway.

IL-1 and IL-1R inhibitors act to inhibit the association of IL-1 and its receptor IL-1R. Upon IL-1 binding downstream tumour-necrosis factor-associated factor (TRAF) TRAF6.

IL-6 and IL-6R inhibitors act to inhibit the association of IL-6 and its receptor IL-6R. Upon IL-6 binding downstream Janus kinase (JAK) in activated and the downstream phosphorylation of signal transducer and activator of transcription 3 (STAT3).

IL-8 and IL-8R inhibitors act to inhibit the association of the IL-8 with its receptor, CXCR1 and/or CXCR2 (IL-8R). Upon IL-8 binding to either receptor it triggers downstream signalling of multiple pathways. IL-8 signalling promotes activation of its primary effectors phosphatidyl-inositol 3-kinase (PI3K) or phospholipase C promoting downstream activation of Akt, PKC, calcium mobilization and/or MAPK signalling cascades.

According to an embodiment of the present invention, an inhibitor of interleukin or the respective receptor comprises an antibody or a fragment thereof, peptide(s), RNA, small molecule or macromolecule and any combination thereof capable of blocking the interaction between said interleukin and the respective receptor. In an embodiment according to the present invention, the IL-1/IL-1R inhibitors are IL-1/IL-1R binding antagonists, which may be antibody or a fragment thereof, peptides(s) or molecule which block the interaction between IL-1 and its receptor IL-1R. The antibody or a fragment thereof, peptide(s) or molecule thereof may bind specifically to IL-1 or to IL-1R for disrupting the interaction between IL-1 and IL-1R and inhibiting the downstream signalling. The anti-IL1/IL-1R antibody may be a chimeric, humanized or monoclonal antibody or any fragment or any molecule thereof. According to the present invention IL-1/IL-1R inhibitor may be any suitable IL-1/IL-1R inhibitor and it is selected based on the required treatment. In an exemplary embodiment according to the invention, anti-IL-1/IL-1R antibody or a fragment thereof, peptide(s) or molecule may be selected any current developmental assets, for example Anakinra (Swedish Orphan Biovitrium) and any combination thereof. These developmental anti-IL-1/IL-1R antibodies or a fragment thereof, peptide(s) or molecules are only examples of currently disclosed and known development antibodies, fragments, peptides and molecules being developed in the field, the present invention is not limited to these.

In an embodiment according to the present invention, the IL-6/IL-6R inhibitors are IL-6/IL-6R binding antagonists, which may be antibody or a fragment(s), peptide(s) or molecule thereof which block the interaction between IL-6 and its receptor IL-6R. The antibody or a fragment thereof, peptide(s) or molecule thereof may bind specifically to IL-6 or to IL-6R for disrupting the interaction between IL-6 and IL-6R and inhibiting the downstream signalling. The anti-IL-6/IL-6R antibody may be a chimeric, humanized or monoclonal antibody or any fragment or any molecule thereof. According to the present invention IL-6/IL-6R inhibitor may be any suitable IL-6/IL-6R inhibitor and it is selected based on the required treatment. In an exemplary embodiment according to the invention, anti-IL-6/IL-6R antibody of a fragment thereof, peptide(s) or molecule may be selected any current developmental assets, for example Tocilizumab (Hoffmann-La Roche SA) and Siltuximab (EUSA Pharmaceuticals Ltd) and any combination thereof. These developmental anti-IL-6/IL-6R antibodies or a fragment thereof, peptide(s) or molecules are only examples of currently disclosed and known development antibodies, fragments and molecules being developed in the field, the present invention is not limited to these.

In an embodiment according to the present invention, the IL-8/IL-8R inhibitors are IL-8/IL-8R binding antagonists, which may be antibody or a fragment thereof, peptide(s) or molecule thereof which block the interaction between IL-8 and its receptor IL-8R. The antibody or a fragment thereof, peptide(s) or molecule thereof may bind specifically to IL-8 or to IL-8R for disrupting the interaction between IL-8 and IL-8R and inhibiting the downstream signalling. The anti-IL-8/IL-8R antibody may be a chimeric, humanized or monoclonal antibody or any fragment or any molecule thereof. According to the present invention IL-8/IL-8R inhibitor may be any suitable IL-8/IL-8R inhibitor and it is selected based on the required treatment. In an exemplary embodiment according to the invention, anti-IL-8/IL-8R antibody, fragment or molecule may be selected any current developmental assets for example Reparixin (Dompe Farmaceutici SpA), AZD-5069 (AstraZeneca Plc), BMS-986253 (Bristol-Myers Squibb Co) and Navarixin (Merck & Co Inc) or any combination thereof. These developmental anti-IL-8/IL-8R antibodies, fragments or molecules are only examples of currently disclosed and known development antibodies or a fragment thereof, peptide(s) and molecules being developed in the field, the present invention is not limited to these.

An agent capable to bind to interferon-alpha/beta receptor (IFNAR) is one which is capable of binding to the receptor and inducing the Tyk2 and Jak1, which results in signal transducer and activator of transcription (STAT).

According to an embodiment of the present invention, an agent which is capable of binding to IFNAR would be any type 1 Interferon (type I IFN) binding agonists, which may be antibody or a fragment thereof, peptide(s) or molecule thereof which binds to the receptor IFNAR inducing the downstream pathways. The antibody, fragment or molecule thereof binds specifically to type I interferon receptor IFNAR and inducing the downstream signalling. The type 1 IFN antibody may be a chimeric, humanized or monoclonal antibody or any fragment or any molecule thereof. According to an embodiment of the present invention, an agent capable of binding to interferon-alpha/beta receptor (IFNAR) is an exogenous type 1 Interferon or an agent capable of inducing similar effects. An exogenous type 1 Interferon includes subtypes of interferon-alpha and interferon-beta. In an embodiment of the present invention, an agent capable of binding to interferon-alpha/beta receptor (IFNAR) comprises interferon alpha or interferon beta. According to an embodiment of the present invention an exogenous type I interferon may be interferon beta-1a or interferon beta-1b. According to the present invention an agent capable of binding to interferon-alpha/beta receptor (IFNAR) is selected based on the required treatment. In an exemplary embodiment according to the invention, an agent capable of binding to IFNAR may be selected any current developmental assets, for example Rebif (Merck and Co) comprising interferon beta-1a, Avonex (Biogen) comprising interferon beta-1a, Betaseron (Bayer) comprising interferon beta-1b and Traumakine (Faron Pharmaceuticals) comprising interferon beta-1a, or any combination thereof. These type 1 IFN drug products are only examples of currently disclosed and known development type I IFN agonists, the present invention is not limited to these.

According to an embodiment of the invention, a method for treating or preventing diseases selected from the group consisting of cancer, infectious diseases, chronic infection, sepsis, severe influenza or corona virus infection and acute respiratory distress syndrome (ARDS), comprises administering to an individual therapeutically effective amount of:
an agent capable of inhibiting CLEVER-1 expression or capable of binding to CLEVER-1, such as anti-CLEVER-1 antibody or fragment thereof, peptide(s), RNA, small molecule or macromolecule and any combination thereof, and
at least one of the following inhibitors/agents:
an anti-IL-1 and/or IL-1R inhibitor, such as an anti-IL-1/IL-1R antibody, fragment or molecule thereof that binds specifically to IL-1 or the receptor(s) IL-1Rα or IL-1Rβ and inhibits the activity of IL-1 by these means,
an anti-IL-6 and/or IL-6R inhibitor, such as an anti-IL-6/IL-6R antibody, fragment or molecule thereof that binds specifically to IL-6 or the receptor (IL-6R) and inhibits the activity of IL-6 by these means, and
an anti-IL-8 and/or IL-8R inhibitor, such as an anti-IL-8/IL-8R antibody, fragment or molecule thereof that binds specifically to a Interleukin 8 (IL-8) or the receptor(s) CXCR1 or CXCR2 and inhibits the activity of IL-8 by these means, and
optionally further an agent capable to bind to Interferon-alpha/beta receptor (IFNAR), such as an exogenous type 1 Interferon.

The present invention may be useful for treating a disease states with an exhausted immune response, which are not responsive to anti-CLEVER-1 agent or interleukin inhibitor(s) and/or their respective receptor(s) or type 1 interferon as single agents. According to an embodiment of the invention, anti-CLEVER-1 agent(s) in combination with interleukin inhibitor(s) and/or their respective receptor(s) and optionally in combination with type 1 interferon is used in treating an individual having diagnosed with a disease state of immune exhaustion relating to cancer, infections, sepsis and ARDS. According to the present invention, anti-CLEVER-1 agent in combination with interleukin inhibitor(s) and optionally in combination with type 1 interferon can be used in treatment or prevention of disease selected from the group consisting of cancer, infectious diseases, chronic infection, sepsis, severe influenza or corona infection, acute respiratory distress syndrome (ARDS).

Infectious diseases are caused by pathogenic microorganisms, such as bacteria, viruses, parasites or fungi; the diseases can be spread, directly or indirectly, from one person to another. Infectious diseases may be caused by viral organisms such as influenza and corona viruses. Inflamed or infected tissue with immune exhaustion can be characterized by high macrophage infiltration and/or low T cell infiltration, or by the elevated expression of checkpoint inhibitors on T cells populations obtained through a blood sample.

According to an embodiment of the present invention, anti-CLEVER-1 agent in combination with interleukin inhibitor(s) and/or their respective receptor(s) and optionally in combination with type 1 interferon is used for treating cancer by reducing malignant tumour growth and/or by inhibiting metastasis formation is applicable to all forms of cancers. Thus, any benign or malignant tumour or metastasis of malignant tumour can be treated. According to an embodiment of the present invention, anti-CLEVER-1 agent in combination with interleukin inhibitor(s) and optionally in combination with their respective receptor(s) and/or type 1 interferon is used for creating an immune response to the infectious pathogen.

The present invention is based on the finding that an increase in plasma/serum interleukins, such as IL-6 and IL-8 with CLEVER-1 inhibition is associated with no anti-tumour response despite achieved immune activation, which has been observed by increase in CD8+ T cells, NK cells and plasma IFNγ. A decrease in interleukins in plasma is associated with tumour shrinkage. The present invention is most valuable for patients having diagnosed with a tumour associated with high expression of IL-6 and/or IL-8, since then the inhibition of CLEVER-1 can convert cold tumours hot and increase the efficacy of immunotherapy in patients, which would not normally respond to such a therapy.

According to an embodiment of the present invention, an individual to be treated having diagnosed an elevation in interleukin levels, typically plasma/serum interleukin levels, such as expression levels of IL-1, IL-6 and/or IL-8 after beginning anti-CLEVER-1 treatment.

In an embodiment of the present invention, expression levels of IL-1, IL-6 and/or IL-8 are measured from a patient in order to decide the need for the concomitant interleukin inhibitor(s) and/or their respective receptor(s) treatment, and also to decide the need for the concomitant type 1 interferon treatment. In an embodiment of the present invention, a method for monitoring patient’s response to anti-CLEVER-1 therapy and evaluating the need for combination therapy, when an agent capable of binding to CLEVER-1 has been administered in a patient, comprises
obtaining a sample from the patient at a first point in time prior to the administration of an agent capable of binding to CLEVER-1 to a patient,
obtaining a sample from the patient at a later point in time after the administration of an agent capable of binding to CLEVER-1 to a patient,
measuring a level of interleukin IL-1, interleukin IL-6 and/or IL-8 from the obtained samples,
comparing the level of IL-1, IL-6 and/or IL-8 measured from the sample obtained at a later point of time to the expression level of IL-1, IL-6 and/or IL-8 measured from the sample obtained at a first point of time, wherein an elevation in interleukin IL-1, IL-6 and/or IL-8 levels is an indication for initiation the concomitant administration of IL-1 inhibitor and/or an inhibitor of the respective receptor, IL-6 inhibitor and/or an inhibitor of the respective receptor, IL-8 inhibitor and/or an inhibitor of the respective receptor, or any combination of thereof. In an embodiment according to the present invention interleukin IL-1, IL-6 and/or IL-8 levels are measured from a blood sample, preferably from a serum sample.

According to an embodiment of the present invention the method further comprises measuring IFNy response, wherein IFNy is measured from the sample obtained at a first point in time prior to the administration of an agent capable of binding to CLEVER-1 to a patient and the sample obtained at a later point in time after the administration of an agent capable of binding to CLEVER-1 to a patient and the measured levels are compared. In an embodiment of the present invention, a decision to start the concomitant interleukin inhibitor(s) and/or their respective receptor(s) treatment, and also to decide the need for the concomitant type 1 interferon treatment, is made after both an elevation of IFNy and an elevation in interleukin IL-1, IL-6 and/or IL-8 levels are observed.

The present invention relates also a treatment method comprising an administration of an agent capable of inhibiting CLEVER-1 expression or capable of binding to CLEVER-1 to a patient in combination with an administration of an inhibitor of interleukin(s) such as IL-1, IL-6 and IL-8 and/or their respective receptors IL-1Ra, IL-1Rb, IL-6R and IL-8R, and optionally further with an administration of an agent capable of binding to interferon-alpha/beta receptor (IFNAR), when patient suffering disease selected from the group consisting of cancer, infectious diseases, chronic infection, sepsis, severe influenza or coronavirus infection and acute respiratory distress syndrome (ARDS) with immune exhaustion. Especially, a treatment method of said combination according to the embodiment of the present invention is valuable when the patient is first treated with anti-CLEVER-1 treatment alone and the patient shows high expression of pro-inflammatory cytokines (IL-1, IL-6, IL-8) and/or shows an increase in the level of circulating interleukins during anti-CLEVER-1 treatment.

In an embodiment according to the present invention, a method of treatment comprising administering to anti-CLEVER-1 agent to a patient and after that measuring interferon-gamma and/or interleukin, such as IL-1, IL-6 and/or IL-8 levels. If the desired response is not observed, the treatment is continued by administering anti-CLEVER-1 agent in combination with an inhibitor of interleukin(s) such as IL-1, IL-6 and IL-8 and/or their respective receptors IL-1Ra, IL-1Rb, IL-6R and IL-8R. The measured interferon-gamma and interleukin values such as IL-1, IL-6 and IL-8 are compared to the values measured from said patient prior to the starting of anti-CLEVER-1 treatment or the values of the previous measurement(s) during the anti-CLEVER-1 treatment. If IL-1, IL-6 and/or IL-8 response is not desired, the efficacy of anti-CLEVER-1 treatment may be improved by administering an inhibitor of interleukin(s) such as IL-1, IL-6 and IL-8 and/or their respective receptors IL-1Ra, IL-1Rb, IL-6R and IL-8R. Further, the response may be improved by administering an agent capable of binding to interferon-alpha/beta receptor (IFNAR).

According to an embodiment the present invention, an agent capable of inhibiting CLEVER-1 expression or capable of binding to CLEVER-1 is administered to an individual prior to the administration of an inhibitor of interleukin and/or an inhibitor of the respective interleukin receptor. According to an embodiment the present invention, an agent capable of inhibiting CLEVER-1 expression or capable of binding to CLEVER-1 is administered to an individual prior to prior to the administration of an agent capable of binding to IFNAR. According to another embodiment of the invention, an agent capable of binding to CLEVER-1 is administrated to the individual simultaneously with an inhibitor of interleukin and/or an inhibitor of the respective interleukin receptor, wherein they may be admixed as a single composition or administered concurrently. In an embodiment of the present invention an agent capable of binding to IFNAR is also administered simultaneously with an agent capable of binding to CLEVER-1 and/or an inhibitor of interleukin and/or an inhibitor of the respective interleukin receptor, wherein they may be admixed as a single composition or administered concurrently. In an embodiment according to the present invention, an agent capable of inhibiting CLEVER-1 expression or capable of binding to CLEVER-1 and an inhibitor of interleukin and/or an inhibitor of the respective interleukin receptor, and optionally also an agent capable of binding to IFNAR may be administered sequentially, wherein at least part of the anti-CLEVER-1 agents are administered prior to an interleukin inhibitor and/or an inhibitor of the respective interleukin receptor and/or an agent capable of binding to IFNAR. Administering may be performed, for example once, a plurality of times, and/or over one or more extended periods.

According to an embodiment of the present invention, an administration of an agent capable of inhibiting CLEVER-1 expression or capable of binding to CLEVER-1 is continued to an individual after the administration of an inhibitor of interleukin(s) and/or an inhibitor of the respective receptor(s), and/or after the administration of an agent capable of binding to interferon-alpha/beta receptor (IFNAR). In an embodiment of the present invention, the patient may be firstly treated with an interleukin inhibitor and/or an inhibitor of the respective interleukin receptor in combination with type 1 IFN, and after notifying that the desired treatment response has not been achieved, the treatment can be continued by administering anti-CLEVER-1 agent(s) in combination with an interleukin inhibitor and/or an inhibitor of the respective interleukin receptor(s) and/or type 1 IFN inhibitor.

According to an embodiment of the present invention, an interleukin inhibitor or an inhibitor of the respective receptor is administrated to an individual prior to an agent capable of binding to IFNAR, or an interleukin inhibitor and/or an inhibitor of the respective receptor is administrated to an individual after an agent capable of binding to IFNAR, where no response is seen after the first treatment. In an embodiment of the present invention, an interleukin inhibitor and/or an inhibitor of the respective receptor is administrated to an individual simultaneously with an agent capable of binding to IFNAR.

“Administering” refers to the physical introduction of a composition comprising said therapeutic agents to an individual, using any of the various methods and delivery systems known to those skilled in the art. The agents to be used in the present invention may be administered by any means that achieve their intended purpose. For example, administration may be oral, inhaled, intravenous, intramuscular, intraperitoneal, intra-tumoral, subcutaneous or other parenteral routes of administration, for example by injection. In addition to the pharmacologically active compounds, the pharmaceutical preparations of said agents preferably contain suitable pharmaceutically acceptable carriers comprising excipients and auxiliaries that facilitate processing of the active agents into preparations that can be used pharmaceutically. The dose chosen should be sufficient to reduce malignant tumour growth and/or inhibit metastasis formation and/or to block the negative regulation of T cells in cancer, chronic infection, infectious diseases or other states of immune exhaustion, e.g. in sepsis and ARDS.

In treatment methods according to the present invention, also any other anticancer agents may be used in addition to an interleukin inhibitor(s) and/or Type 1 interferon and anti-CLEVER-1 agents.

According to an embodiment of the present invention a humanized monoclonal anti-CLEVER-1 antibody is administered in the range of 0.3 - 10 mg/kg, preferably 0.3 mg/kg to 3 mg/kg, according to the patient’s body weight. In an embodiment according to the present invention a humanized monoclonal anti-CLEVER-1 antibody comprises the therapeutic anti-CLEVER-1 antibody bexmarilimab (FP-1305) and it is administered in the range of 0.3 - 10 mg/kg, preferably 0.3 mg/kg to 3 mg/kg, according to the patient’s body weight. Said dose range is preferred also in the presented combination treatment for providing immune stimulation for said diseases. In an embodiment according to the present invention, 0.3 - 10 mg/kg, preferably 0.3 - 3 mg/kg according to the patient’s body weight, of a humanized monoclonal anti-CLEVER-1 antibody, such as bexmarilimab (FP-1305) is used in combination with an inhibitor of interleukin and/or the respective interleukin receptor, and optionally also with an agent capable of binding to interferon-alpha/beta receptor (IFNAR). Typically, a patient to be treated has not shown desired response to anti-CLEVER-1 treatment, such as the therapeutic anti-CLEVER-1 antibody bexmarilimab (FP-1305) treatment alone and/or having diagnosed an elevation in interleukin levels, such as IL-1, IL-6 and IL-8 after beginning anti-CLEVER-1 treatment although IFN-gamma levels are increased and shown response.

### EXPERIMENTAL PART

The following studies are merely illustrative of the principles of the present invention and are not intended to limit the scope of the invention.

### Human Studies on Clever-1 Inhibition for the Treatment of Cancer

CLEVER-1 inhibiting agent, an anti-CLEVER-1 antibody FP-1305 is currently being tested for safety and preliminary efficacy in a Phase I/II study in patient with advanced solid tumors (clinicaltrials.gov NCT03733990: A Study to Evaluate Safety, Tolerability and Preliminary Efficacy of FP-1305 in Cancer Patients (MATINS)).

An anti-CLEVER-1 antibody FP-1305 is a humanized monoclonal CLEVER-1 antibody, previously presented in the Patent Publication WO2017/182705. More precisely, FP-1305 (DSM ACC3361) is a humanized monoclonal immunoglobulin G4_{K} antibody Bexmarilimab (International Nonproprietary Name (INN)) as disclosed in WHO Drug Information, Vol. 33, No.4 (2019) as proposed INN and in WHO Drug Information, Vol. 34, No. 3 (2020), pages 699-700 as recommended INN).

In the present study, first (pre-dose) serum sample taken prior to initiating FP-1305. Second serum sample (post-dose) taken 7 days after beginning FP-1305 treatment, and following at 14 days, 21 days, and 42 days and 63 days from initiating anti-CLEVER-1 treatment. After which tumour progression or regression is evaluated repeating a CT scan that is compared to an existing scan taken before initiating anti-Clever-1 treatment (FIG. 1). Progressive disease (PD) means the cancer is growing. No significant change in tumour size, which is a positive effect in aggressive otherwise non-treatable cancers, such as in the MATINS trial, is labelled as stable disease (SD), and considered good response. Tumour shrinkage is referred to as partial response (PR) according to the RECIST criteria used to evaluate treatment response.

### Plasma/Serum IL6 and IL8 Increase is Associated With Non-Response In Cancer Patients Treated With Anti-CLEVER-1 Antibody (FP-1305)

The anti-CLEVER-1 antibody FP-1305 has begun clinical development in the setting explained above. In this first-in human trial (clinicaltrials.gov NCT03733990) metastatic colorectal cancer, melanoma and ovarian cancer patients that have not been responsive to any available therapy have shown anti-tumour responses. These so far have all been associated with an increase in serum IFNγ levels during treatment (FIG. 1). IFNγ, IL-6 and IL-8 serum levels were measured using multiplex cytokine panel. Surprisingly opposite to the IFNγ response, an increase in IL-6 and IL-8 levels were associated with non-response, i.e. progressive disease. Hence, anti-CLEVER-1 antibody FP-1305 treatment may be improved by administering an inhibitor of interleukin and/or the respective interleukin receptor, and optionally also with an agent capable of binding to interferon-alpha/beta receptor (IFNAR) to a patient for decreasing IL-6 and/or IL-8 levels.

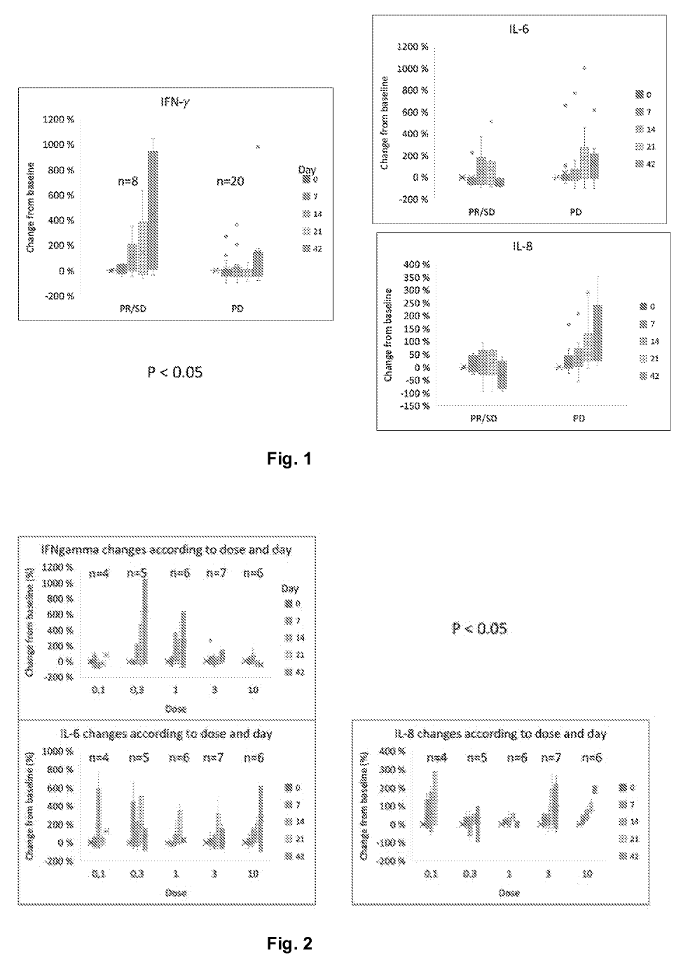

In addition, when comparing different doses, best immune activation measured by IFNy elevation was achieved using doses of FP-1305 ranging from 0.3 mg/kg to 3 mg/kg according to the patient’s body weight. In similar fashion most favourable changes in IL-6 and IL-8 was seen in doses of FP-1305 ranging from 0.3 mg/kg to 3 mg/kg according to the patient’s body weight. The smallest dose 0.1 mg/kg of FP-1305 had no significant immunological changes, while the highest dose 10 mg/kg associated with biggest elevations in IL-6 and IL-8 (FIG. 2). Hence, a dose range of 0.3 - 10 mg/kg, preferably 0.3 mg/kg to 3 mg/kg, according to the patient’s body weight, of the humanized monoclonal anti-CLEVER-1 antibody FP-1305 can be used in combination with an inhibitor of interleukin and/or the respective interleukin receptor, and optionally also with an agent capable of binding to interferon-alpha/beta receptor (IFNAR).

### Anti-CLEVER-1 Reinvigorates an Exhausted Immune System and Helps to Fight Sepsis

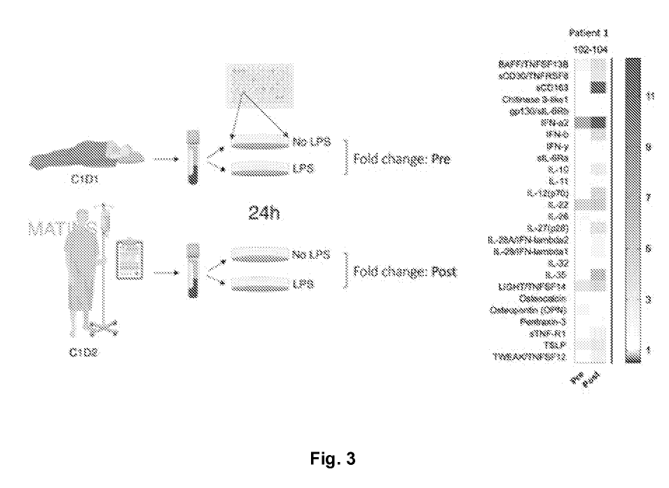

In the on-going anti-CLEVER-1 antibody FP-1305 first-in human trial (clinicaltrials.gov NCT03733990) a colorectal cancer patient (Patient 1 in FIG. 3) with an extremely exhausted immune system was enrolled to receive anti-Clever-1 antibody FP-1395 treatment at a dose of 1 mg/kg according to the patient’s body weight. Complete immune exhaustion prior to receiving anti-CLEVER-1 antibody FP-1305 therapy was seen by giving her peripheral blood cells fragments of bacteria, i.e. lipopolysaccharides (LPS). Her blood cells could not react to the given LPS, meaning that in case of a significant infection her immune system could not generate the required immune response and the infection would most likely lead to her death. Twenty-four hours after receiving the first dose of FP-1305 the LPS experiment was repeated. Now the patient’s peripheral acted normally to the LPS stimulus and generated cytokines and inflammatory signals needed to give raise to an immune response against a foreign pathogen. Subsequently, the patient got sepsis due to cholestasis but was able to survive sepsis because she had received FP-1305. Without the treatment she would have not been able to respond to sepsis appropriately.

### CITED REFERENCES

Mayer-Barber K.D. and Yan B. Clash of the Cytokine Titans: Counter-regulation of interleukin-1 and type I interferon-mediated inflammatory responses. Cellular & Molecular Immunology, 2017, 14 (1) 22-35.

Griffiths MJD et al. Guidelines on the management of acute respiratory distress syndrome. BMJ Open Respiratory Research 2019; 6 (1).

Meduri, GU et al. Persistent elevation of inflammatory cytokines predicts a poor outcome in ARDS. Plasma IL-1 beta and IL-6 levels are consistent and efficient predictors of outcome over time. Chest. 1995 107 (4) 1062- 73.

Bauer TT et al. Comparison of systemic cytokine levels in patients with acute respiratory distress syndrome, severe pneumonia, and controls. Thorax. 2000. 55. 46-52.

Waugh JJD and Wilson C., The Interleukin-8 Pathway in Cancer, Molecular Pathways, 2008,14(21).

Diao B. et al. Reduction and Functional Exhaustion of T Cells in Patients with Coronavirus Disease 2019 (COVID-19). medRxiv preprint https://doi.orq/10.1101/2020.02.18.20024364, posted Feb. 20, 2020.

Dong, Y. et al. CD4⁺ T cell exhaustion revealed by high PD-1 and LAG-3 expression and the loss of helper T cell function in chronic hepatitis B. BMC Immunology. 2019. 20 (27).

Fasoulakis, Z. et al. Interleukins in breast cancer. Cureus. 2018. 10 (11).

Pardoll DM., The blockade of immune checkpoints in cancer immunotherapy, Nature reviews cancer, 2012, 12(4): 252 - 264).

Chen DS, Mellman I. Elements of cancer immunity and the cancer-immune set point. Nature 2017; 541(7637): 321-30.

Donini, C., D’Ambrosio, L., Grignani, G., Aglietta, M. and Sangiolo, D. Next generation immune-checkpoints for cancer therapy. Journal of Thoracic Disease 2018; 10 (Suppl 13): S1581 -S1601.

Hegde PS, Karanikas V, Evers S. The Where, the When, and the How of Immune Monitoring for Cancer Immunotherapies in the Era of Checkpoint Inhibition. Clin Cancer Res 2016;22(8):1865-74 doi 10.1158/1078-0432.CCR-15-1507.

Obeid M, Tesniere A, Ghiringhelli F, Fimia GM, Apetoh L, Perfettini JL, et. al. Calreticulin exposure dictates the immunogenicity of cancer cell death. Nat Med 2007; 13(1):54-61.

Guerriero JL, Sotayo A, Ponichtera HE, Castrillon JA, Pourzia AL, Schad S, et al. Class Ila HDAC inhibition reduces breast tumours and metastases through anti-tumour macrophages. Nature 2017; 543(7645):428-32.

Qian BZ, Pollard JW. Macrophage diversity enhances tumor progression and metastasis. Cell 2010; 141(1):39-51.

Ries CH, Cannarile MA, Hoves S, Benz J, Wartha K, Runza V, et al. Targeting tumor-associated macrophages with anti-CSF-1R antibody reveals a strategy for cancer therapy. Cancer Cell 2014; 25(6):846-59.

Quail DF, Bowman RL, Akkari L, Quick ML, Schuhmacher AJ, Huse JT, et al. The tumor microenvironment underlies acquired resistance to CSF-1R inhibition in gliomas. Science 2016;352(6288): aad3018.

Kzhyshkowska J, Gratchev A, Goerdt S. Stabilin-1, a homeostatic scavenger receptor with multiple functions. J Cell Mol Med 2006;10(3):635-49.

Kzhyshkowska J, Workman G, Cardó-Vila M, Arap W, Pasqualini R, Gratchev A, et al. Novel function of alternatively activated macrophages: stabilin-1-mediated clearance of SPARC. J Immunol 2006;176(10):5825-32.

Karikoski M, Marttila-Ichihara F, Elima K, Rantakari P, Hollmen M, Kelkka T, et al. Clever-1/Stabilin-1 Controls Cancer Growth and Metastasis. Clinical Cancer Research 2014;20(24):6452-64.

1. A combination of therapeutically effective amounts of:
(a) an agent capable of inhibiting CLEVER-1 expression or capable of binding to CLEVER-1, and
(b) an inhibitor of interleukin and/or the respective interleukin receptor, for use in a treatment or prevention of disease selected from the group consisting of cancer, infectious diseases, chronic infection, severe influenza or coronavirus infection, sepsis and acute respiratory distress syndrome (ARDS), wherein the agent capable of inhibiting CLEVER-1 expression or capable of binding to CLEVER-1 is administrated to an individual prior to the administration of an inhibitor of an interleukin(s) and/or an inhibitor of the respective interleukin receptor(s) and an individual to be treated having diagnosed an elevation in interleukin IL-1, IL-6 and/or IL-8 levels after beginning of the administration of said agent capable of inhibiting CLEVER-1 expression or capable of binding to CLEVER-1.

2. The combination for use according to claim 1, wherein the agent capable of inhibiting CLEVER-1 expression or capable of binding to CLEVER-1 is selected from the group consisting of an antibody or a fragment thereof, peptide(s), RNA, small molecule or macromolecule and any combination thereof.

3. The combination for use according to claim 1, wherein the agent capable of inhibiting CLEVER-1 expression or capable of binding to CLEVER-1 comprises the humanized monoclonal anti-CLEVER-1 antibody, preferably bexmarilimab (DSM ACC3361) or bexmarilimab variant or the antibody in a bexmarilimab biosimilar.

4. The combination for use according to claim 1, wherein an inhibitor of interleukin and/or the respective interleukin receptor is selected from the group consisting of IL-1 inhibitor and/or an inhibitor of the respective receptor, IL-6 inhibitor and/or an inhibitor of the respective receptor, IL-8 inhibitor and/or an inhibitor of the respective receptor, or any combination of thereof.

5. The combination for use according to claim 1, wherein an inhibitor of interleukin or the respective interleukin receptor comprises an antibody or a fragment thereof, peptide(s), RNA, small molecule or macromolecule and any combination thereof capable of blocking the interaction between said interleukin and the respective receptor.

6. The combination for use according to claim 1, wherein the combination further comprises an agent capable of binding to interferon-alpha/beta receptor (IFNAR).

7. The combination for use according to claim 6, wherein the agent capable of binding to interferon-alpha/beta receptor (IFNAR) is an exogenous type 1 Interferon.

8. The combination for use according to claim 1, wherein the agent capable of inhibiting CLEVER-1 expression or capable of binding to CLEVER-1 is administrated to an individual simultaneously with an inhibitor of interleukin(s) and/or an inhibitor of the respective receptor(s), and/or simultaneously with an agent capable of binding to interferon-alpha/beta receptor (IFNAR).

9. The combination for use according to claim 1, wherein administration of the agent capable of inhibiting CLEVER-1 expression or capable of binding to CLEVER-1 is continued to an individual after the administration of an inhibitor of interleukin(s) and/or an inhibitor of the respective receptor(s), and/or after the administration of an agent capable of binding to interferon-alpha/beta receptor (IFNAR).

10. The combination for use according to claim 6, wherein an inhibitor of interleukin(s) and/or an inhibitor of the respective receptor(s) is administrated to an individual simultaneously with an agent capable of binding to interferon-alpha/beta receptor (IFNAR).

11. The combination for use according to claim 6, wherein an inhibitor of interleukin(s) and/or an inhibitor of the respective receptor(s) is administrated to an individual after an agent capable of binding to interferon-alpha/beta receptor (IFNAR).

12. The combination for use according to claim 3, wherein the humanized anti -CLEVER-1 antibody bexmarilimab is administered in the range of 0.3 - 10 mg/kg, preferably 0.3 mg/kg to 3 mg/kg, according to the patient’s body weight.

13. A method for monitoring a patient’s response to anti-CLEVER-1 therapy and evaluating the need for combination therapy comprising an inhibitor of interleukin and/or the respective interleukin receptor, when an agent capable of binding to CLEVER-1 has been administered in a patient, the method comprising
obtaining a sample from the patient at a first point in time prior to the administration of an agent capable of binding to CLEVER-1 to a patient,
obtaining a sample from the patient at a later point in time after the administration of an agent capable of binding to CLEVER-1 to a patient,
measuring a level of interleukin IL-1, IL-6 and/or IL-8 from the obtained samples,
comparing the level of IL-1, IL-6 and/or IL-8 measured from the sample obtained at a later point of time to the expression level of IL-1, IL-6 and/or IL-8 measured from the sample obtained at a first point of time, wherein an elevation in interleukin IL-1, IL-6 and/or IL-8 levels is an indication for initiation the concomitant administration of IL-1 inhibitor and/or an inhibitor of the respective receptor, IL-6 inhibitor and/or an inhibitor of the respective receptor, IL-8 inhibitor and/or an inhibitor of the respective receptor, or any combination of thereof.

14. The method according to claim 13, wherein the method further comprises measuring IFNy response, wherein IFNγ is measured from the sample obtained at a first point in time prior to the administration of an agent capable of binding to CLEVER-1 to a patient and the sample obtained at a later point in time after the administration of an agent capable of binding to CLEVER-1 to a patient and the measured levels are compared.

15. The method according to claim 13, wherein the sample is a blood sample, preferably serum sample.
